(19) United States
(12) Patent Application Publication
(10) Pub. No.: US 2010/0006105 A1
(43) Pub. Date: Jan. 14, 2010

(54) APPARATUS AND METHODS FOR OCCLUDING A FALLOPIAN TUBE

(76) Inventors: Phillip J. Carter, Park City, UT (US); Richard J. Linder, Sandy, UT (US); Randall K. Jones, Murray, UT (US); Scott D. Miles, Sandy, UT (US); Clark C. Davis, Holladay, UT (US); Daryl R. Edmiston, Draper, UT (US); DeWayne C. Fox, South Jordan, UT (US)

Correspondence Address: Holland & Hart LLP/Coherex Medical, 60 East South Temple, Suite 2000, Salt Lake City, UT, 84111, US

(21) Appl. No.: 12/169,506

(22) Filed: Jul. 8, 2008

(51) Int. Cl. A61F 6/22; A61B 17/425

(52) U.S. Cl. 128/831; 606/119

(57) ABSTRACT

The present invention is directed to various embodiments of medical devices and methods for occluding a fallopian tube for contraception and permanent sterilization. In one embodiment, the medical device includes an outer member, an inner member and a tissue growth member. The outer member includes an outer surface and an inner surface, wherein the inner surface defines a bore in the outer member. The inner member is configured to be positioned within the bore of the outer member. The tissue growth member is attached to the outer surface of the outer member and is configured to induce tissue growth thereto. With this arrangement, the medical device can be implanted within the fallopian tube and serve as a permanent occluding device therein. If desired, the medical device can be partially removed from the fallopian tube to restore the ability for conception.

### TECHNICAL FIELD

The present invention relates generally to sterilization techniques. More specifically, the present invention relates to devices and methods for occluding a fallopian tube for contraception and permanent sterilization.

### BACKGROUND

The current world population is experiencing explosive growth, potentially creating serious problems in various aspects of society. To minimize and avoid pregnancies, women of reproductive age have relied on various contraception and sterilization techniques. Such techniques typically fall within the categories of physical barriers, drugs and surgery, each of which have proven to be less than satisfactory and in some cases harmful.

For example, conventional physical barriers can include condoms and diaphragms. Such barriers are subject to failure due to breakage, displacement and often are not used consistently so as to totally prevent an eventual conception. Drug strategies, such as the pill, which rely on artificially controlling hormone levels, have been found to be somewhat effective for contraception purposes. However, it has been found that users of the pill for long periods of time suffer from harmful side-effects, some currently known and un-known. Finally, surgical procedures, such as tubal ligation, are the most common for permanent sterilization. Such surgical procedures, however, are extremely expensive, highly invasive and involve the attendant risks of surgery.

Thus, the continuing need for additional safe, low cost, reliable methods of contraception and permanent sterilization, both in developed and less developed countries, is widely recognized. As such, it would be advantageous to develop non-surgical methods and devices for contraception and sterilization. It would also be advantageous for such non-surgical methods and devices to be reversible should an individual's circumstances change after receiving a sterilization procedure.

### BRIEF SUMMARY OF THE INVENTION

The present invention is directed to various embodiments of a medical system, devices and methods for occluding a fallopian tube for contraception and permanent sterilization. In one embodiment, a medical device includes an outer member, an inner member and a tissue growth member. The outer member includes an outer surface and an inner surface, wherein the inner surface defines a bore in the outer member. The inner member is configured to be positioned within the bore of the outer member. The tissue growth member is attached to the outer surface of the outer member and is configured to induce tissue growth thereto. With this arrangement, the medical device can be implanted within the fallopian tube and serve as a permanent occluding device therein. If desired, the medical device can be partially removed to restore the ability for conception by removing the inner member from the outer member to, thereby, expose the bore in the outer member.

In another embodiment, the medical device includes a frame member and a tissue growth member. The frame member includes a substantially flat configuration and is configured to be moved between a narrow constrained position and an expanded position. The tissue growth member is attached to the frame member and is configured to induce tissue growth thereto and occlude the fallopian tube.

In another embodiment, the medical device includes tines configured to assist preventing migration of the device within the fallopian tube. These tines can be positioned on the outer peripheral edges of the frame member or outer member so as to grab onto tissue walls within the fallopian tube. In another embodiment, the frame member includes proximal anchors and distal anchors configured to expand outward and bias walls of the fallopian tube. Such proximal and distal anchors can also include such tines.

In another embodiment, the frame member can include a multi-cellular structure to assist the frame member between the narrow constrained position and the expanded position while maintaining a substantially flat configuration.

In accordance with other embodiments of the present invention, methods of occluding a fallopian tube are provided as well as methods of reversing the occlusion of a fallopian tube. In one embodiment, a method for reversing occlusion of a fallopian tube is provided that includes inserting a medical device having an outer member and an inner member into a fallopian tube and anchoring the outer member of the medical device in the fallopian tube. The medical device is partially removed from the fallopian tube by removing the inner member from the outer member to expose a bore in the outer member

### BRIEF DESCRIPTION OF THE SEVERAL VIEWS OF THE DRAWINGS

To further clarify the above and other advantages and features of the present invention, a more particular description of the invention will be rendered by reference to specific embodiments thereof which are illustrated in the appended drawings. It is appreciated that theses drawings depict only typical embodiments of the invention and are therefore not to be considered limiting of its scope. The invention will be described and explained with additional specificity and detail through the use of the accompanying drawings in which:

FIG. 1 is a perspective view of a medical device for positioning within a fallopian tube, according to an embodiment of the present invention;

FIG. 1(a) is a cross-sectional view of the medical device depicted in FIG. 1 taken along line A;

FIG. 1(b) is a cross-sectional view of a medical device with additional anchors, according to another embodiment of the present invention;

FIG. 1(c) is a cross-sectional view of of a medical device with additional anchors, according to another embodiment of the present invention;

FIG. 2(a) is a cross-sectional view of the medical device of FIG. 1, depicting the medical device at a distal end of a catheter, according to an embodiment of the present invention;

FIG. 2(b) is a cross-sectional view of the medical device of FIG. 1, depicting the medical device being delivered within a fallopian tube prior to being released from the catheter, according to an embodiment of the present invention;

FIG. 2(c) is a cross-sectional view of the medical device of FIG. 1, depicting the medical device being released from the catheter within the fallopian tube, according to an embodiment of the present invention;

FIG. 3 is a cross-sectional view of the medical device of FIG. 1, depicting the medical device positioned within a fallopian tube, with tissue in-growth thereto, according to an embodiment of the present invention;

FIG. 3(a) is a cross-sectional view of the medical device of FIG. 3, depicting a catheter butting up to a proximal side of the medical device prior to removing an inner member from the medical device, according to an embodiment of the present invention;

FIG. 3(b) is a cross-sectional view of the medical device of FIG. 3, depicting a capture member disposed over a portion of the medical device, according to an embodiment of the present invention;

FIG. 3(c) is a cross-sectional view of the medical device of FIG. 3, depicting the capture member capturing the inner member via an inner sheath, according to an embodiment of the present invention;

FIG. 3(d) is a cross-sectional view of the medical device of FIG. 3, depicting the capture member withdrawing the inner member from the medical device, according to an embodiment of the present invention;

FIG. 3(e) is a cross-sectional view of the medical device of FIG. 3, depicting the catheter and inner member withdrawing from the fallopian tube and leaving an outer member of the medical device in the fallopian tube, according to an embodiment of the present invention;

FIG. 4 is a perspective view of a medical device, depicting the medical device having a single rod therein, according to another embodiment of the present invention;

FIG. 4(a) is a cross-sectional view of the medical device depicted in FIG. 4 taken along line A, according to an embodiment of the present invention;

FIG. 4(b) is a side view of the medical device of FIG. 4, depicting another embodiment of the present invention whereinthe inner member is removed from the outer member;

FIG. 5 is a cross-sectional view of another embodiment of a medical device, depicting additional features for an inner member and the tissue growth member;

FIG. 5(a) is a cross-sectional view of the inner member depicted in the device of FIG. 5, according to an embodiment of the present invention;

FIG. 6 is a cross-sectional view of another embodiment of a medical device for positioning within a fallopian tube, depicting an inner member having a tubular configuration;

FIG. 7 is a cross-sectional view of another embodiment of a medical device for positioning within a fallopian tube;

FIG. 8 is a cross-sectional view of another embodiment of a medical device for positioning within a fallopian tube;

FIG. 9 is a cross-sectional view of another embodiment of a medical device for positioning within a fallopian tube;

FIG. 10 is a cross-sectional view of another embodiment of a medical device for positioning within a fallopian tube, depicting an O-ring arrangement between the inner and outer member;

FIG. 11 is a cross-sectional view of another embodiment of a medical device for positioning within a fallopian tube, depicting a valve arrangement between the inner and outer member;

FIG. 12 is a cross-sectional view of the medical device of FIG. 11 with the inner member removed;

FIG. 13 is a cross-sectional view of another embodiment of a medical device, depicting the medical device having a membrane;

FIG. 14 is a cross-sectional view of the medical device of FIG. 13, depicting the medical device in the fallopian tube with tissue in-growth to the medical device and the membrane removed therefrom;

FIG. 15 is a cross-sectional view of a medical device with a membrane at both ends of the device, according to another embodiment of the present invention;

FIG. 16 is a cross-sectional view of another embodiment of a medical device, depicting the medical device having additional anchors with another outer and inner member arrangement;

FIG. 17 is a cross-sectional view of another embodiment of a medical device, depicting an outer and inner member arrangement with multiple seals therebetween;

FIG. 18 is a side view of another embodiment of a medical device within a fallopian tube, depicting the medical device having a circular tissue growth member;

FIG. 19 is a side view of another embodiment of a medical device within a fallopian tube, depicting the medical device having an elongated tissue growth member;

FIG. 20 is a top view of a medical device for positioning in a fallopian tube, depicting a multi-cellular frame and tissue growth member arrangement, according to one embodiment of the present invention;

FIG. 20(a) is a end view of the medical device of FIG. 20, depicting the tissue growth member being out-of-plane with the frame;

FIG. 21 is a top view of another embodiment of the medical device for positioning in a fallopian tube, depicting another structure for the tissue in-growth member;

FIG. 22 is a perspective view of a medical device for positioning in a fallopian tube, depicting another frame structure, according to another embodiment of the present invention;

FIG. 23 is a perspective view of a medical device for positioning in a fallopian tube, depicting an oval frame configuration and tissue in-growth member, according to one embodiment of the present invention;

FIG. 23(a) is an end view of the medical device depicted in FIG. 23;

FIG. 24 is a perspective view of a variation of the medical device of FIG. 23, according to another embodiment of the present invention;

FIG. 25 is a perspective view of a medical device for positioning in a fallopian tube so that a portion of the device sits in the ostium adjacent to the uterus, according to an embodiment of the present invention;

FIG. 26 is a side view of another embodiment of a medical device for positioning in a fallopian tube so that a portion of the device sits in the ostium adjacent the uterus;

FIG. 27 is a perspective view of a medical device for positioning within a fallopian tube, depicting a frame having a multi-planar configuration, according to another embodiment of the present invention;

FIG. 28 is a top view of a medical device, depicting anchors with single beam members, according to yet another embodiment of the present invention;

FIG. 28(a) is a top view of the medical device of FIG. 28 within a fallopian tube;

FIG. 29 is a top view of a medical device in an unconstrained configuration, according to an embodiment of the present invention;

FIG. 29(a) is a top view of the medical device of FIG. 29 within a fallopian tube;

FIG. 30 is a top view of another medical device for positioning within a fallopian tube, depicting a dual anchoring system, according to an embodiment of the present invention;

FIG. 31(a) is a cross-sectional view of the medical device of FIG. 30 taken along section 31a;

FIG. 31(b) is another embodiment of the device depicted in FIG. 31(a);

FIG. 32(a) is an enlarged partial view of an anchor end portion as indicated in FIG. 30, according to one embodiment of the present invention;

FIG. 32(b) is an enlarged partial view of another embodiment of the device depicted in FIG. 32(a);

FIG. 33(a) is an enlarged partial view of an anchor portion as indicated in FIG. 30, according to one embodiment of the present invention;

FIG. 33(b) is an enlarged partial view of another embodiment of the device depicted in FIG. 33(a);

FIG. 33(c) is an enlarged partial view of another embodiment of the device depicted in FIG. 33(a);

FIG. 34 is a cross-sectional view of a medical device, according to a further embodiment of the present invention; and

FIG. 34(a) is a cross-sectional view of the medical device of FIG. 34, depicting the device in an expanded position, according to an embodiment of the present invention.

### DETAILED DESCRIPTION OF THE INVENTION

There is disclosed herein various embodiments of the present invention, including medical systems, devices and methods, that employ a non-surgical procedure for implanting a medical device into a fallopian tube to, thereby, occlude the tube for contraception. For example, such contraception procedure can be readily employed by a physician, such as a gynecologist, within a doctor's office or non-surgical setting. The medical device can be inserted transcervically within the fallopian tube, positioned as desired and anchored. Such placement and anchoring of the medical device will initially substantially occlude the fallopian tube and provide contraception. Over time, the medical device will secure itself within the fallopian tube with tissue in-growth to the medical device for permanent sterilization. In one embodiment, the medical device can be implanted within the fallopian tube and, if desired, a portion of the medical device can be removed to enable the sterilization procedure to be reversible and, thereby, restore the ability for conception.

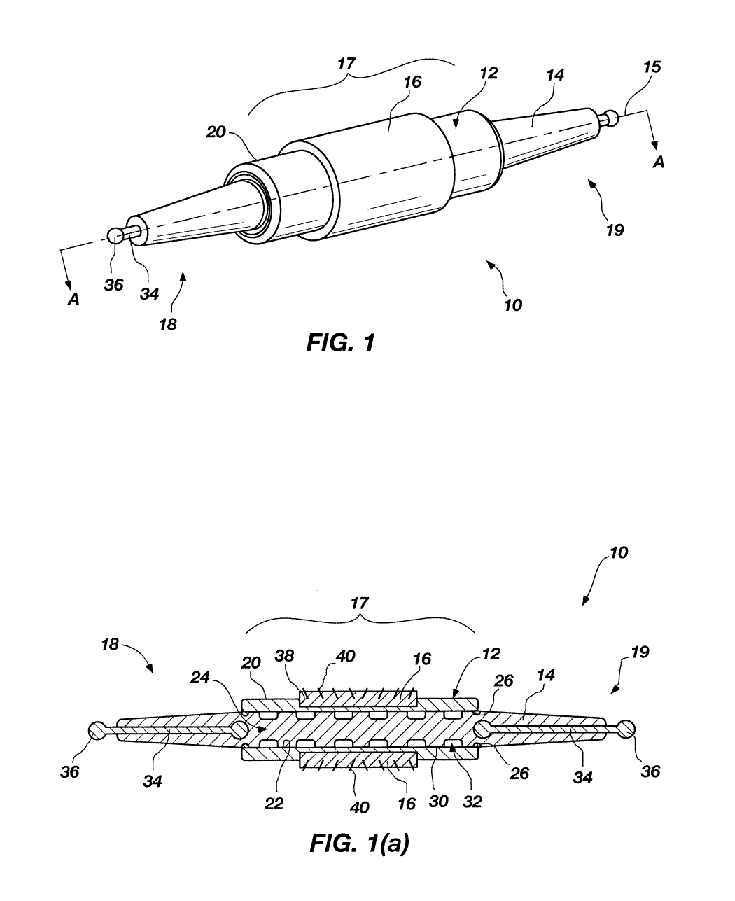

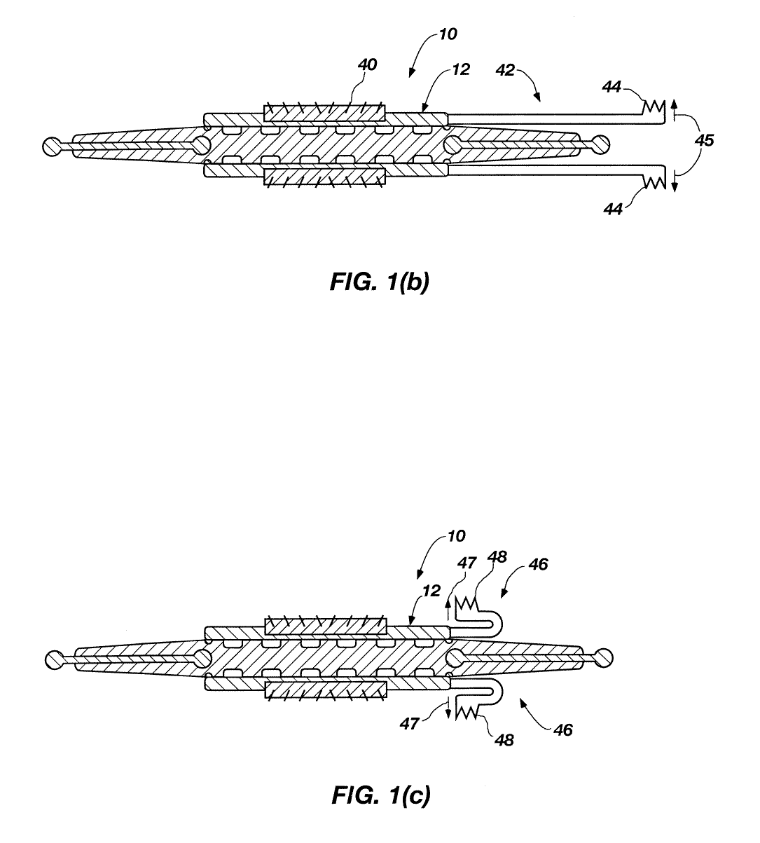

Referring first to FIGS. 1 and 1(a), a medical device 10 can include an outer member 12, an inner member 14 and a tissue growth member 16. Such medical device 10 can include a proximal side 18 and a distal side 19 with a central portion 17 therebetween and can define a longitudinal axis 15 extending through the medical device 10. The outer member 12 can include an outer surface 20 and an inner surface 22 defining a longitudinal bore 24 therethrough. The outer member 12 may exhibit, but is not limited to, a substantially cylindrical or tubular configuration. The outer surface 20 and inner surface 22 of the outer member 12 may exhibit a lateral cross-section (i.e., a cross-section taken substantially perpendicular to the longitudinal axis 15) having surfaces that are substantially circular shaped. The inner surface 22 can also include one or more protrusions 26 extending therefrom. Such protrusions 26 can extend from the proximal side and/or the distal side of the inner surface 22 of the outer member 12. Further, the protrusions 26 can extend from the inner surface 22 radially in, for example, a ring like fashion or a segmented fashion. The outer member 12 can be a rigid or semi-rigid member made from a polymeric and/or metallic type of material, such as, polypropylene, polyester, PEEK, Teflon, titanium, stainless steel, copper, copper alloys or NiTi, or any composite, combination or alloy thereof, or any other suitable material compatible with the human anatomy. Such outer member can be formed utilizing any suitable manufacturing technique known to one of ordinary skill in the art, such as injection molding, casting or machining.

The inner member 14 can include a central portion with proximal and distal portions (i.e., on the proximal and distal sides, respectively) extending longitudinally from opposite sides of the central portion. Such proximal and distal portions can include a tapered configuration extending from the central portion. The central portion of the inner member 14 can be sized and configured with an outer diameter to fit, in a snug manner, within the bore 24 defined in the outer member 12 and can be formed from a flexible or resilient material, such as an elastomeric material. The central portion of the inner member can include ribs 30 that define channels 32 extending radially over a portion of the central portion. Such ribs 30 can be sized and configured to provide multiple sealing portions against the inner surface 22 of the outer member 12. As with the outer member 12, the inner member 14 can be an elastomeric material, such as polyurethane, silicon, PTFE or Teflon, or any other suitable biocompatible material known to one of ordinary skill in the art.

The inner member 14 can also include one or more rods 34 disposed along the longitudinal axis 15 of the inner member 14. As depicted, the inner member can include a rod 34 that extends within each of the proximal and distal portions of the inner member 14. Furthermore, the rod 34 in each of the proximal and distal portions can extend from each respective proximal and distal end with a holding element 36 or ball like structure. Each rod 34 can also include a ball or holding element on the opposite end of such rod 34, sized and configured to maintain the rod within the inner member 14. With this arrangement, the one or more rods 34 can be sized and configured to provide stiffness and enhanced stability to the elastomeric inner member 14. In addition, the one or more rods 34 can serve as a marker to allow a physician to view the medical device 10 under fluoroscopy or any other x-ray system or even under an ultra-sound system. As such, the rods 34 can be formed of radio-opaque materials, such as tantalum, gold or platinum or alloys thereof, or any other suitable materials that can be viewable.

The tissue growth member 16 can be formed, but is not limited to, over a central portion of the outer surface 20 of the outer member 12 in a cylindrical or tubular configuration. Further, such tissue growth member 16 can be attached or formed within a recessed portion 38 at the central portion 17 of the outer member 12. In one embodiment, the tissue growth member 16 can expand slightly larger than the outer diameter of the outer member 12 once the medical device 10 is released form the catheter within the fallopian tube to, thereby, assist the prevention of migration therein. Such tissue growth member 16 can be formed of a porous material, such as foam, mesh, fabric, felt or any other suitable material having a porous structure such that the tissue growth member is sized and configured to induce tissue growth to the medical device. Further, the tissue growth member 16 can be formed of a polymeric or metallic material, such as polyurethane, Teflon, polyester, silicon, Dacron, titanium, stainless steel, NiTi, copper or copper alloys, or composites, combinations or alloys thereof, or any other suitable material, such as a drug induced substance alone or in combination with the above, to induce tissue growth as known to one of ordinary skill in the art.

In one embodiment, as shown in FIG. 1(a), the medical device 10 can include anchors 40. The anchors 40 can extend from the outer member 12 and/or the tissue growth member 16. The anchors 40 can be tines sized and configured to grab or provide resistance against the wall of the fallopian tube and be oriented in a predetermined directional configuration so as to provide resistance from migrating proximally and distally through the fallopian tube. In one embodiment, the anchors 40 can be formed within the tissue growth member 16 such that the tissue growth member 16 can include a porous structure and include a sharp tine structure. Such sharp tine structure formed integrally with the tissue growth member 16 can include tines in either the predetermined directional configuration or a random directional configuration. In another embodiment, the anchors 40 can be sized and configured to be retractable. As known to one of ordinary skill in the art, the anchors 40 can be formed of similar materials set forth for the tissue growth member 16.

FIG. 1(b) discloses another embodiment of the medical device depicted in FIG. 1, however, in this embodiment, the medical device 10 can include one or more anchors 42 extending from the outer member 12. Such anchors 42 can be in addition to or instead of the anchors 40 of the embodiment previously described with respect to FIG. 1(a). The anchors 42 of this embodiment can extend longitudinally and distally from the outer member 12. Such anchors 42 can include an attachment end and a free end, the attachment end attached to a distal end of the outer member 12. In another embodiment, the anchors can extend proximally of the device. The anchors 42 can include tines 44 that extend laterally or transverse at a free end of the anchors 42. Further, the anchors 44 can be configured to self-expand with a force 45 in a lateral direction to effect anchoring of the device into or against the tissue of the fallopian tube (not shown). In this manner, the tines 44 extending laterally can bias against the wall of the fallopian tube and further assist the medical device 10 from self migrating within the fallopian tube. The anchors of this embodiment are, in one embodiment, formed of a metallic material, such as Nitinol, or they may be formed of some other suitable metallic or polymeric material, as known to one of ordinary skill in the art.

FIG. 1(c) discloses another embodiment of anchors 46, extending from the medical device 10, that can be in addition to or instead of the anchors 40 depicted in FIG. 1(a). In this embodiment, the medical device 10 can include anchors 46 extending from a distal end of the outer member 12, wherein the anchors can include a self-energizing structure. Such anchors 46 can extend partially distally and partially proximally with a u-shaped configuration, wherein a free end of the anchors 46 is extending proximally. Similar to the previous embodiment, the anchors 46 can be configured to self expand with an outward force 47 toward the tissue of the fallopian tube. Further, the anchors 46 can include tines 48 at a free end thereof that face the tissue of the fallopian tube. The anchor configuration of this embodiment provides for a self-energizing feature in that, if the medical device 10 moves proximally, then the anchors 46 of this embodiment will provide additional resistance to such movement via the u-shaped configuration. As will be appreciated by one of ordinary skill in the art, the anchors 46 can be formed of similar materials as the anchors of the previously described embodiment.

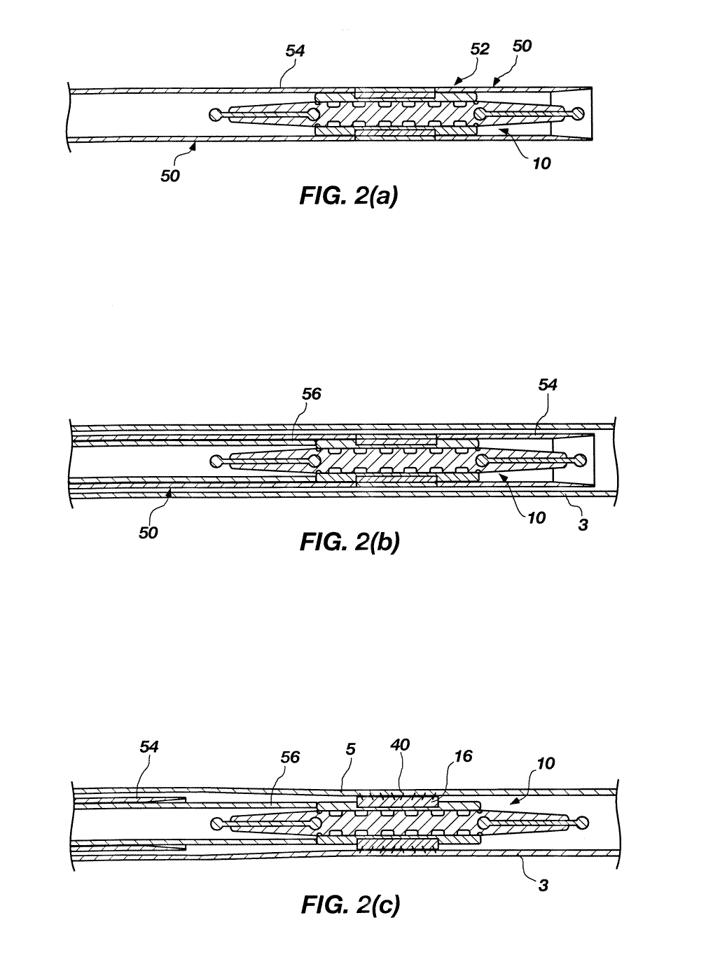

Referring now to FIGS. 2(a) through 2(c), there is disclosed an embodiment for delivering the medical device 10 depicted in FIG. 1 within a fallopian tube 3. In particular, FIG. 2(a) discloses the medical device 10 within a distal end 52 of a catheter 50 prior to insertion within the fallopian tube 3. Such catheter 50 can include an outer sheath 54 and an inner sheath 56 (see FIG. 2b) extending therethrough. FIG. 2(b) discloses the catheter 50 positioned in a desired location within the fallopian tube 3. Prior to deployment of the medical device 10, the inner sheath 56 can be positioned against a proximal end of the outer member 12 of the medical device 10. As illustrated in FIG. 2(c), the outer sheath 54, disposed over the medical device, is pulled back while the inner sheath 56 holds the medical device 10 in a substantially stabilized position. Once the outer sheath 54 is sufficiently pulled from the medical device 10, the catheter 50 can be withdrawn to leave the medical device 10 exposed within the fallopian tube 3. In this manner, the medical device 10 can self anchor to the wall 5 of the fallopian tube 3 with the expansion of the tissue growth member 16(which may include a porous foam structure) and the anchors 40 lodging in the wall 5.

It is also contemplated, in another embodiment, that the medical device can be attached to the delivery system with tethers to readily facilitate re-sheathing the device if it is determined the device is not favorably positioned within the fallopian tube. Once favorable positioning of the device is determined, the medical device can then be detached from such tethers once the outer sheath is withdrawn from the medical device to, thereby, leave the device exposed and anchored within the fallopian tube.

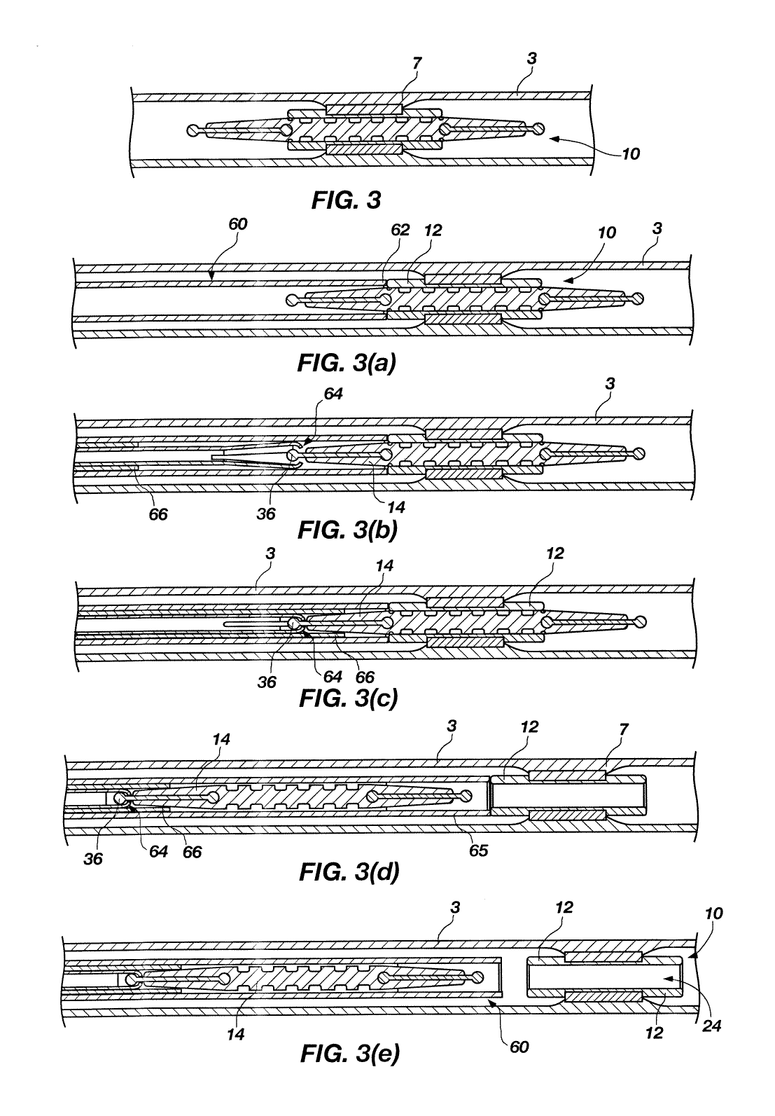

FIG. 3 discloses the medical device 10 after being delivered in the fallopian tube 3 and permanently anchored and attached therein with tissue in-growth 7 to the medical device 10. FIGS. 3(a) through 3(e) disclose one embodiment for removing a portion of the medical device 10 from the fallopian tube 3 to expose the bore 24 of the outer member 12, depicting a method for reversing the procedure of occluding the fallopian tube and restoring the ability of conception. In particular, FIG. 3(a) discloses a distal end 62 of a catheter 60 positioned adjacent and against the proximal end of the outer member 12 of the medical device 10 within the fallopian tube 3. Once the catheter is suitably positioned, a capture member 64 can be moved distally and positioned over a proximal end or the holding element 36 of the inner member 14 (as depicted in FIG. 3(b)), after which, an inner sheath 66 can proceed distally over the capture member 64 to, thereby, close the capture member 64 over the holding element 36 of the inner member 14 (as depicted in FIG. 3(c)). Once the capture member 64 is clamped around the holding element 36, and as shown in FIG. 3(d), the inner sheath 66 and the capture member 64 may be withdrawn proximally while an outer sheath 65 of the catheter 60 remains against the proximal end of the outer member 12. Such outer sheath 65 provides support and leverage while withdrawing the inner member 14 from the outer member 12 so that the anchored outer member 12 substantially maintains its tissue growth 7 integrity within the fallopian tube 3. Once the inner member 14 has been fully captured and removed from the outer member 12, the catheter 60 can be withdrawn from the fallopian tube 3, as depicted in FIG. 3(e). With the inner member 14 removed from the medical device 10, and with the outer member 12 maintained intact with the fallopian tube 3, the bore 24 of the outer member is exposed to allow clear passage along the length of the fallopian tube 3, thereby, restoring the ability for conception. In this manner, the procedure for occluding the fallopian tube can be reversed.

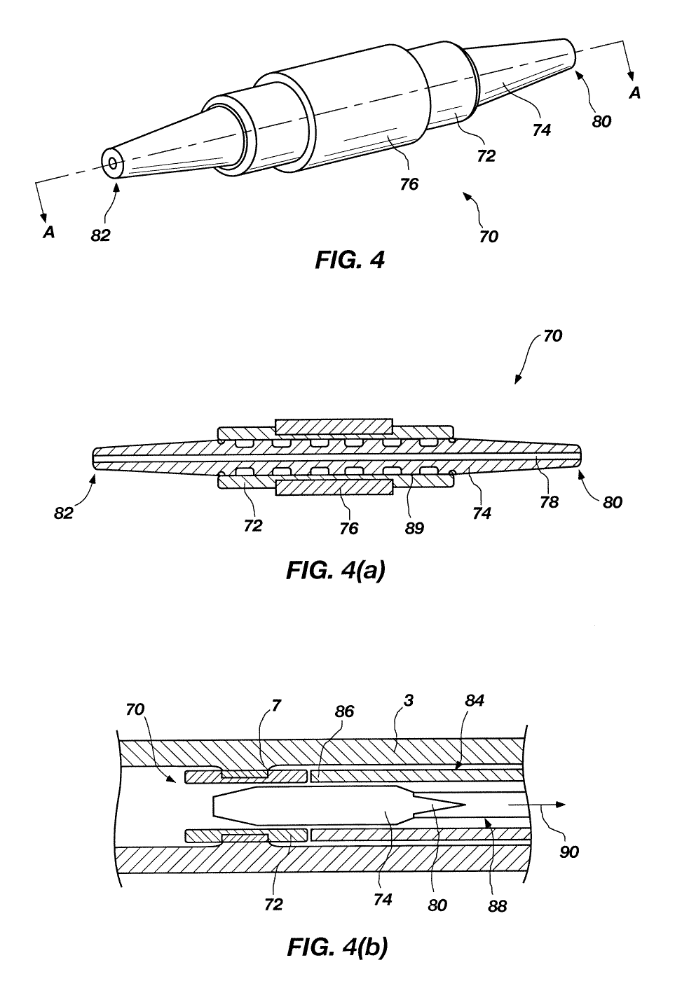

FIGS. 4 and 4(a) disclose a medical device 70, according to another embodiment of the present invention, that includes an outer member 72, an inner member 74 and a tissue growth member 76. This embodiment is similar to previously described embodiments, except in this embodiment, the inner member 74 includes one rod 78 that extends substantially the entire length of the inner member 74. Further, the one rod can be sized and configured to sit flush with the proximal end 80 and distal end 82 of the inner member 74. As illustrated in FIG. 4(b), the medical device of this embodiment can also be employed to provide a reversible procedure. Similar to the reversible procedure depicted in FIGS. 3(a) through 3(e), FIG. 4(b) discloses a distal end 86 of a catheter 84 abutting to the proximal end of the outer member 72 with a clamping device 88 configured to clamp to the proximal end 80 of the inner member 74. Once clamped to the inner member 74, a pulling force 90 can be applied to the inner member 74 with the distal end 86 of the catheter 84 abutting against the outer member 72 to provide leverage and, thereby, minimize potential tearing of the tissue growth 7 between the fallopian tube 3 and the implanted medical device 70. Further, when applying the pulling force to the inner member 74, the diameter of any elastomeric material from which the inner member 74 may be formed is reduced due to stretching to, thereby, break the seal between the ribs 89 (see FIG. 4a) and the inner surface of the outer member 72. In this manner, the inner member 74 can be removed from the implanted medical device to, thereby, employ a reversible procedure.

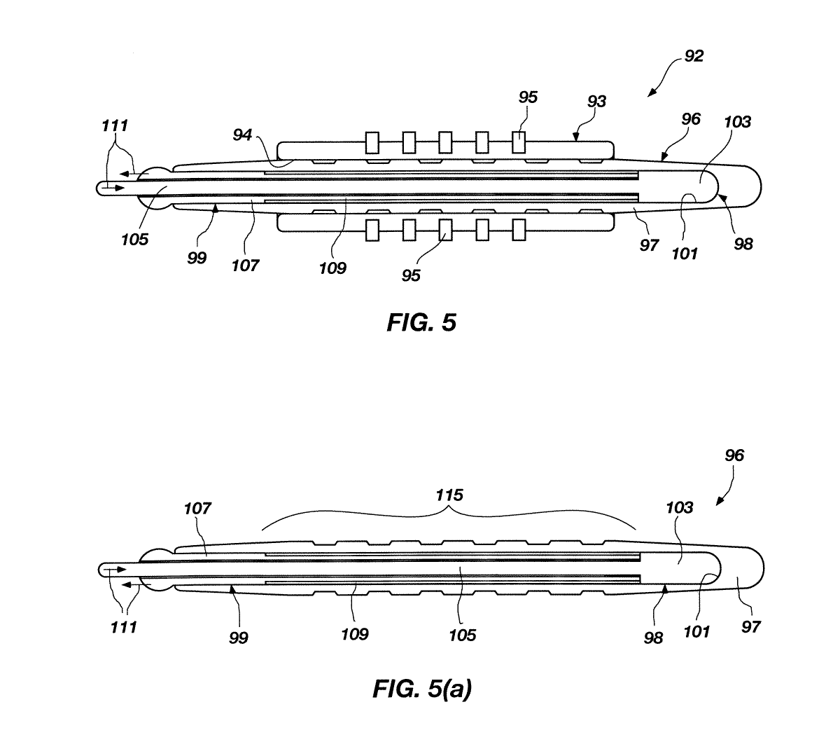

FIGS. 5 and 5(a) disclose another medical device 92, according an embodiment of the present invention. Similar to previous embodiments, the medical device 92 includes an outer member 93 with a tissue growth member 95 formed thereon, the outer member 93 defining a bore 94 sized and configured to receive an inner member 96. However, in this embodiment, the inner member 96 can be configured to be readily inserted into an exposed bore 94 of an outer member 93 that has previously had an inner member removed. In other words, this embodiment provides an inner member 96 that can re-occlude the fallopian tube.

The inner member 96 of this embodiment can include an elastomeric member 97, a first rigid member 98 and a second rigid member 99. The elastomeric member 97 can include a hole 101 defined longitudinally therein with the first and second rigid members 98 and 99 each positioned within the hole 101 of the elastomeric member 97. The first rigid member 98 can include an end portion 103 and an elongated middle portion 105. The second rigid member 99 can include a collar portion 107 and a sleeve portion 109. The end portion 103 of the first rigid member 98 is configured to be secured to, and positioned within, an end or bottom of the hole 101 of the elastomeric member 97 with the elongated middle portion 105 configured to be sized and configured to fit within both the sleeve portion 109 and the collar portion 107 with a semi-loose or moveable arrangement. The collar portion 107 of the second rigid member 99 can be configured to be secured to an inner surface, at a proximal portion of the hole 101 within the elastomeric member 97, with the sleeve portion 109 sized and configured with an outer diameter smaller than the diameter of the hole 101 in the elastomeric member 97. With this arrangement, a longitudinal force 111 can be applied between the proximal ends of the first rigid member 98 and the second rigid member 99 to facilitate minimizing the diameter of the elastomeric member 97. Such longitudinal force 111 can be applied through a catheter (not shown) such that the proximal end of the first rigid member 98 and the second rigid member 99 are moved closer together so as to stretch a central portion 115 of the elastomeric member 97 to minimize the diameter and facilitate such removal and/or insertion of the inner member 96 from and to the outer member 93, respectively. As will be appreciated by one of ordinary skill in the art, the elastomeric member 97 can be formed from a polymeric material or any other suitable materials, such as previously set forth regarding the inner member in previously described embodiments. Further, the rigid member of this embodiment can be formed from a polymeric or metallic material, similar to the rigid materials set forth regarding previously described embodiments.

In another embodiment, the tissue growth member 95 can comprise one or more tissue growth members. That is, the tissue growth member 95 can include, but is not limited to, multiple ring-like configurations disposed around the outer member 93. The medical device 92 can also include anchors (not shown) to stabilize the medical device within the fallopian tube to enable in-growth of tissue with the tissue growth member. The anchors can be extensions from the outer member 93 or extensions from the tissue growth member, as set forth in previously described embodiments.

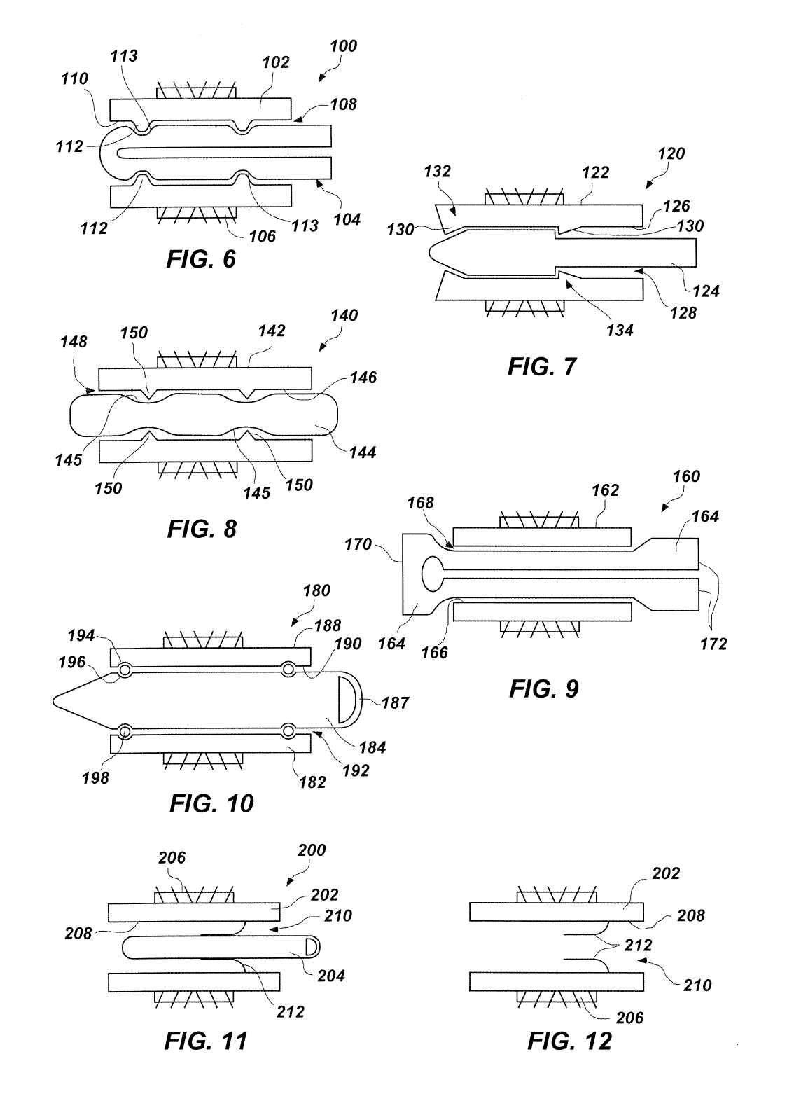

Referring now to FIGS. 6 through 9, there is disclosed various embodiments of a medical device, providing similar function and features disclosed in the previous embodiments, that facilitate a reversible procedure for occluding a fallopian tube. These various embodiments each include an outer member having a tissue growth member attached thereto and an inner member configured to be removable from the outer member. Such inner member can be flexible and/or resilient made from an elastomeric material. Also, the outer member and/or tissue growth member can include anchors or tines to assist anchoring the device within the fallopian tube to substantially prevent migration of the medical device.

More specifically, FIG. 6 discloses a medical device 100 including an outer member 102 with a tissue growth member 106 attached to a central portion of the outer member 102 and an inner member 104 sized and configured to be disposed within a central bore 108 defined in the outer member 102. The outer member 102 includes an inner surface 110, defining the central bore 108, that can include protrusions 112 that can extend from the inner surface 110. Such protrusions 112 can extend radially along the inner surface 110 of the outer member 102 and can be configured to constrict or restrain the inner member 104 from self migrating from the outer member 102. The inner member 104 can be sized and configured to be in a tubular configuration (shown in cross-section having a closed end in FIG. 6) within the central bore 108 of the outer member 102 with one end closed-off. Further, the inner member 104 can include recesses 113 to correspond with the protrusions 112 to further prevent self migration. Such tubular configuration can facilitate ready removal of the inner member 104 by applying a pulling force to one free end (or both ends) of the proximal end of the inner member with, for example, a catheter and clamping device arrangement, such as depicted in FIG. 4(b).

FIG. 7 discloses another embodiment, similar to the previous embodiment, of a medical device 120 for occluding a fallopian tube that facilitates a reversible procedure. In this embodiment, the outer member 122 includes protrusions 130 on the inner surface 126 defining, at least partially, the bore 128 in the outer member 122. Such protrusions 130 can be located, for example, at a distal portion 132 of the inner surface 126 and an intermediate portion 134 of the inner surface 126 and can be configured to contain the inner member 124 from migration. The inner member 124 can be formed of an elastomeric or flexible material to readily facilitate removal of the inner member, as previously set forth in the previous embodiments.

FIG. 8 discloses still another embodiment of a medical device 140 for occluding a fallopian tube that facilitates a reversible procedure. In this embodiment, similar to the outer member depicted in FIG. 6, the outer member 142 includes protrusions 150, extending from the inner surface 146 defining the bore 148 of the outer member 142. The protrusions 150 can include a ring type structure and can be configured to constrain the inner member from self migrating from the outer member 142. The inner member 144 can include recesses 145 configured to correspond to the protrusions 150 to further prevent self migration of the inner member 144. The inner member 144 in this embodiment is elastomeric and extends through the length of the bore.

FIG. 9 discloses another embodiment of a medical device 160, wherein the inner surface 166 of the outer member 162, defining the bore 168, does not include protrusions. In this embodiment, the inner member 164 is sized and configured such that the inner member 164 extends through the bore 168 at a distal end 170 and a proximal end 172 of the outer member 162. Due to the sizing of the inner member 164 and outer member 162, the inner member 164 expands beyond the diameter of the bore 168 to substantially prevent migration of the inner member 164. Further, in this embodiment, the inner member 164 can include a tubular configuration similar to that disclosed for the inner member of FIG. 6. Although not shown, the inner member 164 and outer member 162 can also include the recess/protrusion arrangement, as shown in the previous embodiments, to further prevent the inner member from self migrating.

FIG. 10 discloses another embodiment of a medical device 180 for occluding a fallopian tube that facilitates a reversible procedure. Similar to previously described embodiments, the medical device 180 of this embodiment can include an outer member 182, an inner member 184 and a tissue growth member 186, the tissue growth member being disposed around an outer surface of a central portion of the outer member 182. Such outer member 182 can also include anchors 185 extending therefrom. However, in this embodiment, the inner member 184 can include, but is not limited to, a rigid or semi-rigid material, such as, without limitation, a polymeric and/or metallic type of material similar to the materials previously set forth for the outer member. Furthermore, the medical device 180 can include O-rings 198, or similar structure, configured to be disposed within recesses 194 defined in the inner surface 190 of the bore 192 of the outer member 182. The O-rings 198 can be formed of an elastomeric material, such as EPDM, urethane, fluoro polymer, silicone or polyurethane, or any other suitable material. The inner member 184 can include recesses 196 formed radially in the outer surface of inner member configured to correspond with the O-rings 198. Such O-rings 198 can be configured to be maintained in the recesses 196 of the inner member 184 or the recesses 194 of the outer member 182. In this manner, the inner member 184 is contained within the outer member 182 via the O-ring/recess arrangement and can further readily allow the inner member 184 to be removed if desired via the holding element 187 by utilizing similar methods previously set forth.

FIGS. 11 and 12 disclose another embodiment of a medical device 200 for occluding a fallopian tube that facilitates a reversible procedure. Similar to previously described embodiments, there is disclosed an outer member 202 and an inner member 204 with a tissue growth member 206 attached to the outer member 202. However, in this embodiment, there is a valve member 212 that is positioned within a bore 210 defined by an inner surface 208 of the outer member 202. Such valve member 212 is sized and configured to maintain the inner member 204 within the outer member 202 to, thereby, provide a medical device 200 that occludes the fallopian tube. Using, for example, a method such has been previously set forth, the inner member 204 can be readily removed if desired. Once removed, the valve member 212 can be configured to be maintained in an open position, as depicted in FIG. 12. In this manner, the medical device 200 of this embodiment facilitates a reversible procedure after occluding the fallopian tube.

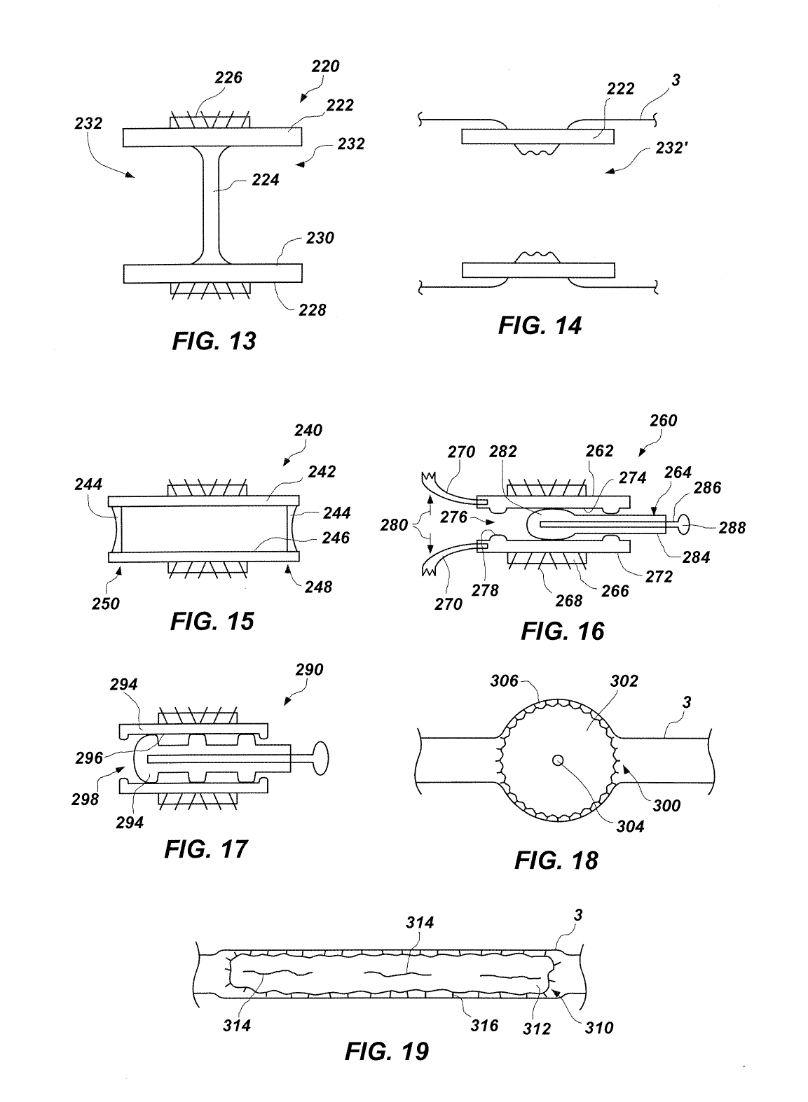

FIGS. 13 and 14 disclose still another embodiment of a medical device 220 that employs a reversible procedure after closing a fallopian tube. This embodiment includes an outer member 222 with a tissue growth member 226 attached to an outer surface 228 at a central portion of the outer member 222. In this embodiment, the outer member 222 can include an inner surface 230 defining partial bores 232 (such as blind holes) each extending longitudinally into the outer member 222 with a membrane 224 extending transversely between the partial bores 232. That is, the membrane 224 can extend transverse to a longitudinal axis of the outer member 222. In this embodiment, the membrane 224 can be an inner member. Such an inner member or membrane 224 can be configured to prevent matter from moving through the fallopian tube. If a reversible procedure is desired, the membrane 224 can be removed (as depicted in FIG. 14) by a burrowing procedure to expose bore 232′ extending longitudinally through the outer member 222 and to facilitate substantially clear access and communication through the fallopian tube 3.

FIG. 15 discloses another embodiment or variation of the embodiment depicted in FIG. 13, except in this embodiment the medical device 240 includes an outer member 242 defining a bore 246 therein with multiple membranes 244 in a dual cap-like configuration. In this embodiment, the multiple membranes 244 can be an inner member. Such multiple membranes 244 are depicted as extending across the bore 246 at the proximal end portion 248 and distal end portion 250 of the outer member 242. Multiple membranes 246 may provide additional measures for ensuring sterilization and blocking passage through the fallopian tube. As previously set forth, if it is desired to reverse this procedure to restore the ability for conception, the membranes can simply be removed through, for example, a burrowing procedure.

FIG. 16 discloses another embodiment of a medical device 260 of the present invention. In this embodiment, the medical device 260 includes an outer member 262 with anchor members 268 extending therefrom and an inner member 264 configured to be positioned at least partially within the outer member 262 and removable therefrom. The outer member 262 includes an outer surface 272 and an inner surface 274 defining a bore 276 extending longitudinally through the outer member 262. The inner surface 274 can include protrusions 278 sized and configured to maintain the inner member 264 within the outer member 262. Similar to that which has been previously set forth, the outer surface 272 of the outer member 262 can also include a tissue growth member 266. The tissue growth member 266 can be attached and formed to the outer surface 272 at a central portion of the outer member 262. As previously noted, such outer member 262 can also include anchors 268 or tines extending therefrom, which can extend from the tissue growth member 266. Further, the outer member 262 can include distal anchors 270 extending from a distal portion of the outer member 262. Such distal anchors 270 can bias in an outward direction 280 configured to anchor into the fallopian tube wall. The distal anchors 270 can be formed of similar materials as the anchors of the previously described embodiments. The inner member 264 can include a distal inner portion 282 and a proximal inner portion 284 and can be formed of an elastomeric material. The distal inner portion 282 can be sized and configured to fit in a sealed manner within the bore 276 with the surface of the distal inner portion 282 in contact with the inner surface 274 of the outer member 262. The inner member 264 can also include a rod 286 extending from the distal inner portion 282 to the proximal inner portion 284 of the inner member 264. The rod 286 can include a ball 288 or other structure formed at the proximal end thereof sized and configured to facilitate removing the inner member 264 from the outer member 262 if desired, similar to that previously set forth in FIGS. 3(a) through 3(e).

FIG. 17 discloses another embodiment of a medical device 290, similar to previously described embodiments, except in this embodiment the medical device 290 includes an inner member 294 that can include multiple sealing or contact surfaces with the inner surface 296, defining the bore 298, of the outer member 292. Such multiple contact or sealing surfaces are configured to provide redundant seals and further protection from allowing anything to pass through the bore 298 of the outer member 292. As in previous embodiments, the inner member 294 can be readily removed from the outer member 292 if desired.

FIG. 18 discloses another medical device 300 according to an embodiment of the present invention. In particular, in this embodiment, the medical device 300 can include a tissue growth member 302 having one or more markers 304 disposed therein. The tissue growth member 302 can be configured to be placed in a constrained state, while being delivered, and then self expands when positioned and released within the fallopian tube 3. The tissue growth member 302 can be sized and configured to expand to a greater size than the diameter of the fallopian tube 3 so that the tissue growth member 302 can become self anchoring while also having the ability to induce tissue growth to and through the tissue growth member 302. The tissue growth member 302 can be a porous material or any suitable material, for example, foam. It is also contemplated that the tissue growth member 302 can include metallic and/or polymeric material therein that can include structure for anchors 306 or tines to assist stabilizing and anchoring the device within the fallopian tube 3 and prevent migration of the device 300. The tissue growth member 302 of this embodiment can be formed from similar materials previously set forth for the tissue growth member of previously described embodiments.

FIG. 19 discloses another embodiment of a medical device 310, with similar features of the device depicted in FIG. 18, according to the present invention. In this embodiment, there is disclosed a medical device 310 that can include a tissue growth member 312 with one or more markers 314 therein. The tissue growth member 312 can include a worm like structure, elongated and flexible, to extend partially or along substantially the full length of the fallopian tube 3. The tissue growth member 312 can be sized and configured to expand to, or slightly larger than, a diameter size of the fallopian tube 3. In this embodiment, the tissue growth member 312, due to the elongated length of the tissue growth member, can also be sized and configured to expand slightly smaller than the diameter of the fallopian tube and still substantially maintain a position within the fallopian tube 3. The tissue growth member 312 can include, as in the previous embodiment, a porous material, for example, foam, and can include metallic and/or polymeric material with structure for anchors 316 or tines to assist stabilizing the device within the fallopian tube 3. The tissue growth member 312 of this embodiment can be formed from similar materials previously set forth for the tissue growth member of previously described embodiments.

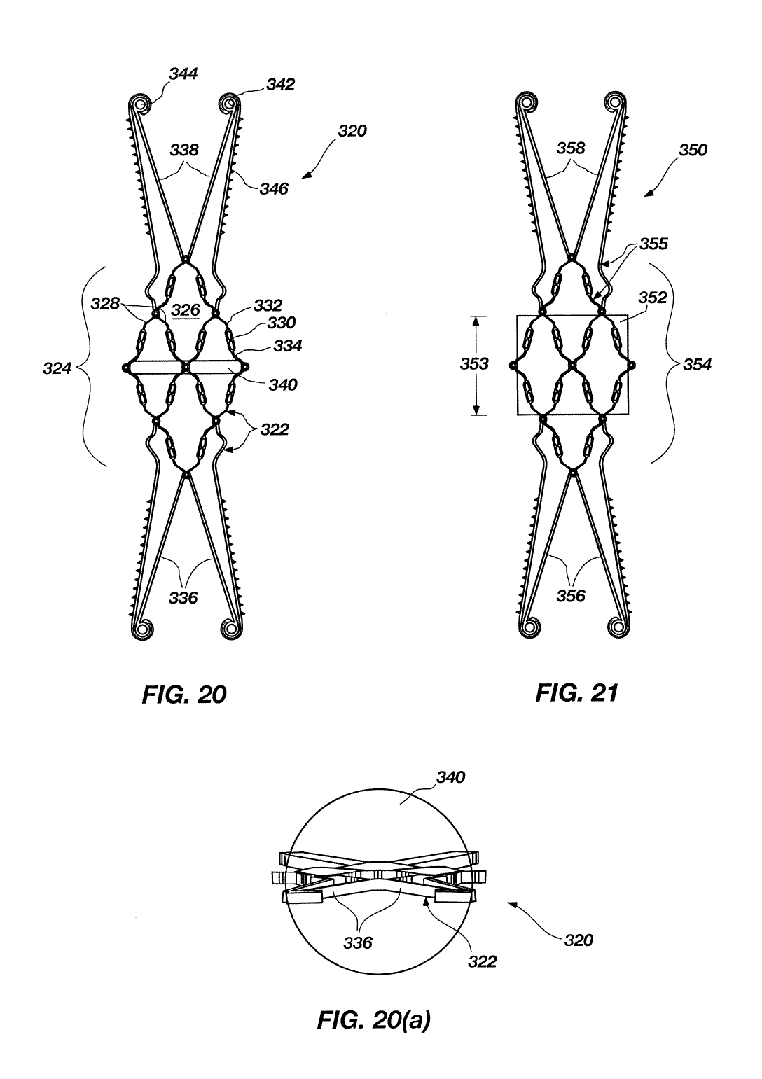

Referring to FIG. 20, there is disclosed another embodiment of a medical device 320 configured to be positioned and anchored within a fallopian tube. In one embodiment, the medical device 320 includes a frame 322 having a substantially flat configuration. Such frame 322 can be configured to self expand in a manner that facilitates or assists in self anchoring within the fallopian tube. Prior to deployment within the fallopian tube, the medical device 320 is configured to be disposed in a constrained position within a distal end of a catheter (not shown). The distal end of the catheter can then be positioned favorably within the fallopian tube, after which, the catheter is moved proximally while the medical device 320 remains substantially stationary. As the catheter moves proximally and the medical device is exposed, the medical device 320 can deploy by self expanding and anchoring itself within the fallopian tube. If it is determined that the medical device 320 is not positioned and anchored satisfactorily, the medical device can readily be re-captured within the catheter. The medical device can then be re-positioned and re-deployed until acceptable positioning is employed. The medical device can then be detached from lines or tethers or any other means for detachment or release.

As previously set forth, the medical device 320 includes a frame 322 that can be a substantially flat configuration. Such substantially flat configuration can be substantially planar or can be substantially flat-like or substantially flat with a bowing effect. Such bowing effect can occur, but is not limited to, by being constrained with portions bowed while in an anchored position within the fallopian tube or while in a constrained position, such as while in the catheter.

The frame 322 can include a central portion 324 with proximal anchors 336 and distal anchors 338 extending therefrom. The central portion 324 can include a multicellular configuration. In one embodiment, the multicellular configuration can include, for example, four cells. Each cell 326 can be defined, at least in part, by one or more side walls 328, for example, four side walls. Further, each cell 326 can be defined by one or more side walls 328 which may be common to an adjacent cell 326. Such side walls 328 can include rigid portions 330 and flexure portions 332. The rigid portions 330 can include a geometry or structure that maintains the side walls in predetermined positions and can provide rigidity to stabilize the flexure portions 332 in predetermined positions. The flexure portions 332 can include tapered portions 334 to facilitate the side walls 328 to flex and expand in predetermined positions and desired configurations. Further, such flexure portions and rigid portions can be configured to minimize stresses and strains within the frame member 322 to increase the structural integrity and cycles the frame member 322 can be moved between the constrained and expanded position.

The proximal anchors 336 and distal anchors 338 can extend from the most proximal and distal cell, respectively, of the central portion 324. The proximal anchors 336 can each extend from portions (for example, end portions) of a side wall 328 of the most proximal cell. Likewise, the distal anchors 338 can each extend from portions of the most distal cell of the central portion 324. The proximal anchors 336 and distal anchors 338 can each respectively extend proximally and distally with marker openings 342 defined at each respective proximal and distal end. Such marker openings 342 can include a radio-opaque material to serve as a marker 344, such as tantalum, gold or platinum or any other suitable material, for purposes of viewing the position of the medical device while within the fallopian tube.

The medical device 320 can be configured to self expand to, in part, facilitate anchoring the device within the fallopian tube. To further assist such anchoring, the outer sides of the proximal anchors 336 and distal anchors 338 can include tines 346 to grab or cause friction against the fallopian tube wall. In this manner, once the medical device 320 is deployed within the fallopian tube, the self expanding feature with the tines 346 substantially prevents migration of the device within the fallopian tube. Further, such self expansion of the medical device (in conjunction with the substantially flat configuration of the frame) pushes the fallopian tube wall outward and pulls a portion of the wall above and below the device (or adjacent the flat or planar sides of the frame 322) inward or closer together and, more importantly, in contact with the tissue growth member 340.

Referring now to the tissue growth member 340, such a tissue growth member 340 can extend laterally along the central portion 324 of the frame 322. As illustrated in FIGS. 20 and 20(a), the tissue growth member 340 extends both laterally and above and below the medical device in an out-of-plane fashion. The tissue growth member 340 can include an oval or rounded periphery that is sized and configured to snugly fit within the fallopian tube so as to contact the wall of the fallopian tube and promote tissue in-growth and, thus, permanent attachment. In this manner, the frame of the medical device 320 includes structural features that enable the device to self expand and, therefore, anchor itself within the fallopian tube. By so anchoring the medical device 320, the medical device also can include the tissue growth member 340 that can enhance tissue growth therein and attach itself to the fallopian tube wall. Such tissue growth member can be formed of a porous material, such as foam or foam like material, or a mesh or woven type material sized and configured to induce tissue growth to and through the tissue growth member. Such material for the tissue growth member can be formed from any of the materials, or any other suitable material, set forth for the tissue growth member in the previous embodiments.

The frame member 322 can be formed of a material that facilitates self-expansion, such as a Nitinol material, that can be formed by laser cutting or any other suitable method, such as etching, ribbon fabrication, crimping, stamping or combinations thereof. It is also contemplated that the frame member 322 can be formed to be manually expandable, in which the frame can be formed of a polymeric and/or metallic type material, such as, polypropylene, polyester, PEEK, Teflon, titanium, stainless steel, copper or copper alloys, or any composite, combination or alloy thereof, or any other suitable material compatible with the human anatomy. Further, it is also contemplated that the frame member 322 (and the tissue growth member 340) can be formed of a bio-resorbable material, such as polylactide, polyglycolide, poly-L-lactide, poly-DL-lactide, and various combinations thereof or any other suitable bio-resorbable material.

FIG. 21 illustrates another embodiment of the medical device 350 of the present invention. In this embodiment, the tissue growth member 352 is sized larger than in the previous embodiment. In particular, the tissue growth member 352 can include a greater depth 353 than the depth of the previous embodiment. In one embodiment, the tissue growth member 352 can extend over a majority of the central portion 354 of the frame 355, or extend over an amount that will suitably fill a portion of the fallopian tube without substantially bending, buckling or folding on itself. Further, similar to the previous embodiment, the tissue growth member 352 of this embodiment can include a height similar to the height (or distance extending out-of-plane) as depicted in FIG. 20(a). The tissue growth member 352 can overlay or cover the central portion 354 of the medical device 350 with a range of approximately 10% to 100%, and in one embodiment, 20% to 90%. In another embodiment, the tissue growth member 352 can cover a portion of the proximal anchors 356 and/or distal anchors 358.

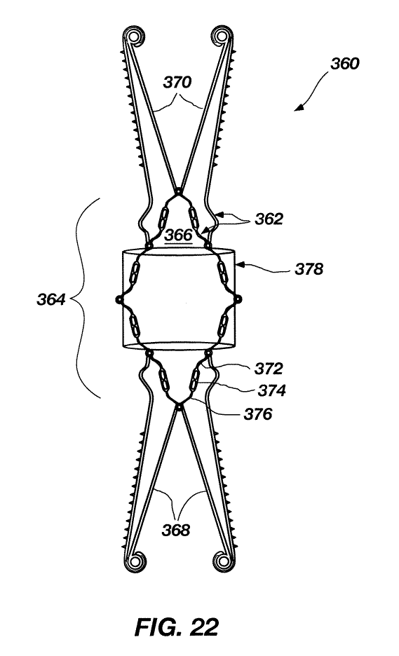

FIG. 22 illustrates another embodiment of the medical device 360 of the present invention. In this embodiment, the medical device 360 is similar to the previous two embodiments, except the medical device in this embodiment includes a frame 362 with a central portion 364 having a single cell 366. The frame 362 can include proximal anchors 368 and distal anchors 370 respectively extending proximally and distally from the central portion 364. The central portion 364 can include side walls 372 defining the single cell 366. Such side walls 372 can include rigid portions 374 and flexure portions 376 sized and configured to facilitate a self expanding structure. Further, similar to the previous embodiments, the medical device 360 can include a tissue growth member 378 sized and configured to induce tissue in-growth when positioned within the fallopian tube.

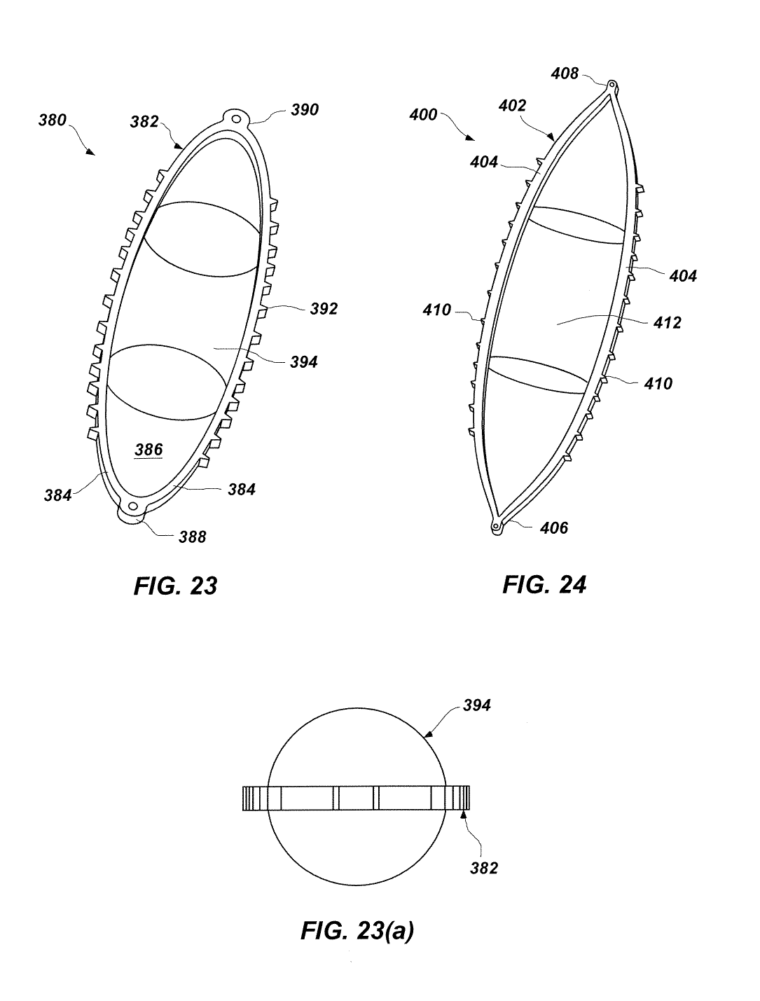

FIG. 23 depicts another embodiment of the medical device 380 of the present invention. In this embodiment, the frame member 382 of the medical device 380 can include side walls 384 defining a single cell 386 extending between a proximal end portion 388 and a distal end portion 390 with an oval shaped configuration. The outer periphery of the side walls 384 can include tines 392 or small anchors. Such tines 392 can be sized and configured to grab or resist migration when positioned within the fallopian tube. Similar to the previously described embodiments, the frame member 382 can also be sized and configured to be placed in a constrained position, while within the catheter prior to deployment, and an unconstrained position to facilitate a self expanding structure to be placed within the fallopian tube and resist migration. The medical device 380 can also include a tissue growth member 394 positioned and attached between the proximal and distal end portions 388 and 390 and opposing side walls 384 within the single cell 386.

With reference to FIGS. 23 and 23(a), as in the embodiments previously set forth, the frame member 382 can include a substantially flat configuration. As such, the tissue growth member 394 can be sized and configured such that the tissue growth member 394 can extend out-of-plane with respect to the frame member 382. In one embodiment, the tissue growth member 394 can include a cylinder type structure. Such tissue growth member 394 can be any suitable size that can be constrained, along with the frame member 382, within a catheter and then self expand when released. Although the tissue growth member 394 is depicted as a cylinder shaped member, the invention is not limited to such structure. That is, the tissue growth member 394 can be any suitable shape or structure that can be constrained within a catheter and then self expand when deployed with the frame member 382 so as to provide an initial effective barrier and, then over time, facilitate tissue growth and provide a permanent barrier.

FIG. 24 discloses another embodiment, similar to previously described embodiments, of a medical device 400. In this embodiment, the medical device 400 includes similar features as that depicted for the medical device in FIG. 23, except in this embodiment the frame member 402 can include opposing side walls 404 extending to a more narrow or acute profile at the proximal end portion 406 and distal end portion 408. Such acute profile can provide and readily facilitate moving between a narrow constrained position to an expanded unconstrained position. The acute profile may be accomplished, for example, by joining the opposing side walls 404 to define a desired angle (e.g., an acute angle) therebetween. As in the previous embodiment, the frame member 402 can include tines 410, a substantially flat configuration and a tissue growth member 412 attached to the frame member 402. Such tissue growth member 412 can extend out-of-plane or beyond the substantially flat configuration of the frame member 402 in a similar manner as depicted by the medical device in FIG. 23(a).

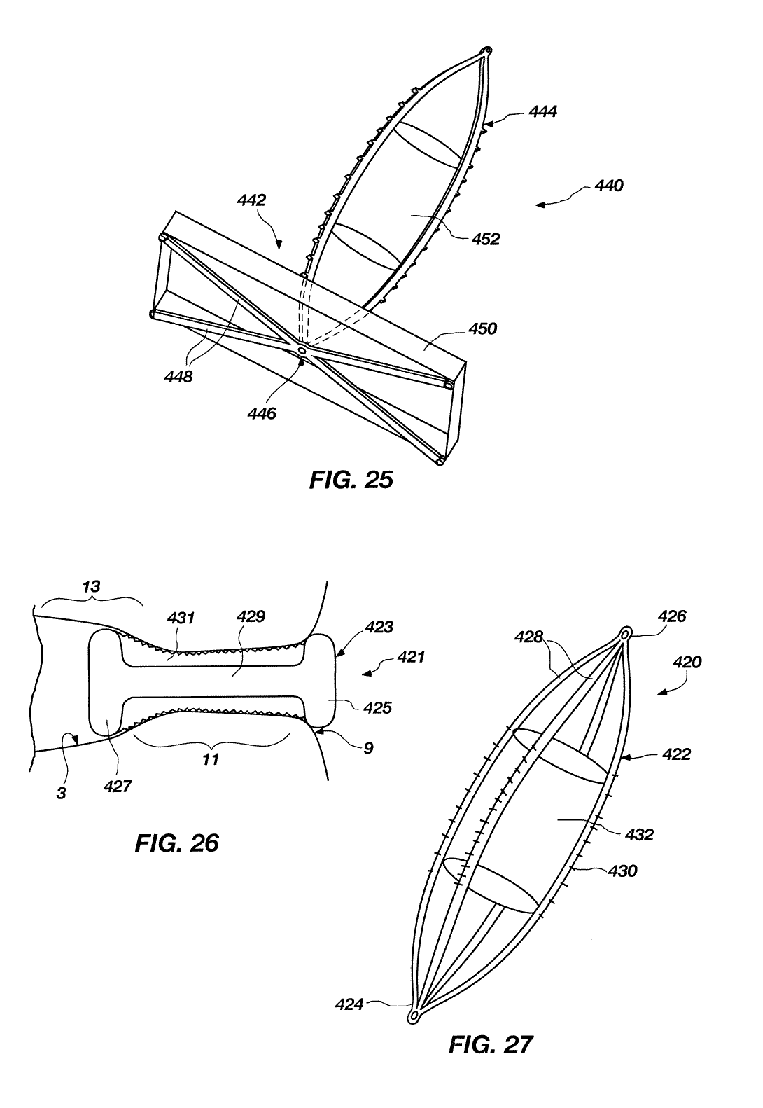

FIG. 25 discloses another embodiment of the medical device 440 of the present invention. In this embodiment, the medical device 440 includes a frame member 441 defining a proximal portion 442 and a distal portion 444, of which the distal portion 444 is similar to the medical device depicted in FIG. 24. However, in this embodiment, the proximal end of the distal portion 444 is interconnected to the proximal portion 442 of the medical device 440. The distal portion 444 is sized and configured to be substantially disposed within the fallopian tub while the proximal portion 442 of the medical device 440 is sized and configured to be disposed over the ostium or entrance into the fallopian tube from the uterus. In particular, the proximal portion 442 of the medical device 440 can be employed as a lid or covering to the ostium and can further facilitate the prevention of migration of the medical device further into the fallopian tube. Further, the combination of the proximal portion 442 and distal portion 444 can be sized and configured so that the proximal portion 442 to act as a gauge sized and configured for proper placement of the distal portion 444 of the medical device 440 within the fallopian tube.

The proximal portion 442 of the medical device can include an attachment portion 446 to the distal portion 444 of the medical device. Such attachment portion can include extension members 448 configured to extend from the attachment portion 446 and sized and configured to provide a skeleton or framework to lay or cover the ostium. The extension members 448 can extend within a common plane to each other or be sized and configured to substantially conform to a wall surface surrounding the ostium within the uterus. In one embodiment, the extension members 448 can include a proximal tissue growth member 450 attached thereto so as to induce tissue growth. In another embodiment, the proximal tissue growth member 450 can be in the form of a membrane on one or both sides of the extension members 448. Such membrane can be configured to limit particular types of fluid into the fallopian tube. The distal portion 444 of the medical device 420 can also include a distal tissue growth member 452 to assist occluding and anchoring within the fallopian tube and induce tissue growth. With this arrangement, the distal portion 444 of the device can be positioned within the fallopian tube with the distal tissue growth member 452 to occlude the tube while the proximal portion 442 serves as a lid or anchor to stabilize the device from unfavorable migration and substantially prevent passage of sperm through the fallopian tubes.

FIG. 26 discloses a medical device 421, according to another embodiment of the present invention. In this embodiment, the medical device 421 can include a frame 423 that can include a solid rigid or semi-rigid structure having, for example, a dog-bone configuration. In another embodiment, the frame can include a stent-like structure that can include self expanding features. In particular, the medical device 421 can include a proximal portion 425 and a distal portion 427 with an elongated central portion 429 therebetween, each of such portions having a generally circular outer periphery (i.e., when viewed in cross-section as taken substantially transverse to a longitudinal axis extending from the proximal portion 425 through the distal portion 427). Further, the proximal portion 425 can include, in cross-section, a larger diameter or dimension (e.g., cross-sectional area) than the central portion 429. Similarly, the distal portion 427 can include, in cross-section, a larger diameter or dimension (e.g., cross-sectional area) than the central portion 429. The central portion 429 can include a tissue growth member 431 attached thereto sized and configured to cover substantially the entire central portion 429. In one embodiment, the tissue growth member 431, in addition to the central portion 429, can extend over the distal portion 427 and/or proximal portion 425 of the medical device 421. The tissue growth member 431 can be formed of a resilient and porous material. The tissue growth member 431 can include, but is not limited to, a thickness or an outer diameter or dimension (when fully expanded), in cross-section, similar to that of the proximal portion 425 and/or the distal portion 427 of the frame 423. With this arrangement, the central portion 429 of the medical device 421 can be positioned within the fallopian tube 3 at a narrow region 11 thereof with the proximal portion 425 adjacent the ostium 9 and the distal portion 427 adjacent a wide region 13 of the fallopian tube 3. In this manner, the proximal and distal portions 425 and 427 are outside the narrow region 11 of the fallopian tube 3 to act as anchors with the central portion 429 contacting tissue at the narrow region of the fallopian tube 3 to incite tissue in-growth thereto. Further, the tissue growth member 431 can have a larger dimension than the inner diameter of the fallopian tube 3 (at the narrow region 11) and, therefore, due to the tissue growth member 431 being a resilient structure such tissue growth member will bias against the tissue of the fallopian tube 3. It is also contemplated that the frame 423 can include additional anchors extending from the frame 423 and/or the tissue growth member 431 to further prevent the medical device from self migration in the fallopian tube.

FIG. 27 discloses another embodiment of a medical device 420 of the present invention. In particular, this embodiment can include a frame member 422 having a multi-planar configuration, that can include, but is not limited to, a frame similar to the substantially planar frame disclosed in FIG. 24, but in a dual frame configuration. Such frame member 422 can include a proximal frame end 424 and distal frame end 426 with multiple frame portions 428 extending longitudinally therebetween. Each of the proximal frame end 424 and the distal frame end 426 serve as a junction for ends of the multiple frame portions 428. The frame portions 428 can be configured to move between a constrained position configured to be positioned within a catheter and an expanded position configured to self-anchor within the fallopian tube. In the expanded configuration, the frame portions, collectively, provide a bulge or bulb that can serve to self anchor within the fallopian tube. Further, the frame portions 428 can include anchors 430 or tines extending from an outer periphery of the frame portions 428 and can be located at an intermediate portion (or frame portions that collectively provide the bulge or bulb) of the frame portions 428. Such anchors 430 with the self-expanding frame portions 428 assist in substantially preventing migration of the medical device 420 within the fallopian tube. Furthermore, the medical device 420 can include a tissue growth member 432 that can be disposed within an interior of the multiple frame portions 428. The tissue growth member 432 can be sized and configured to induce tissue in-growth to permanently attach itself to the fallopian tube wall.

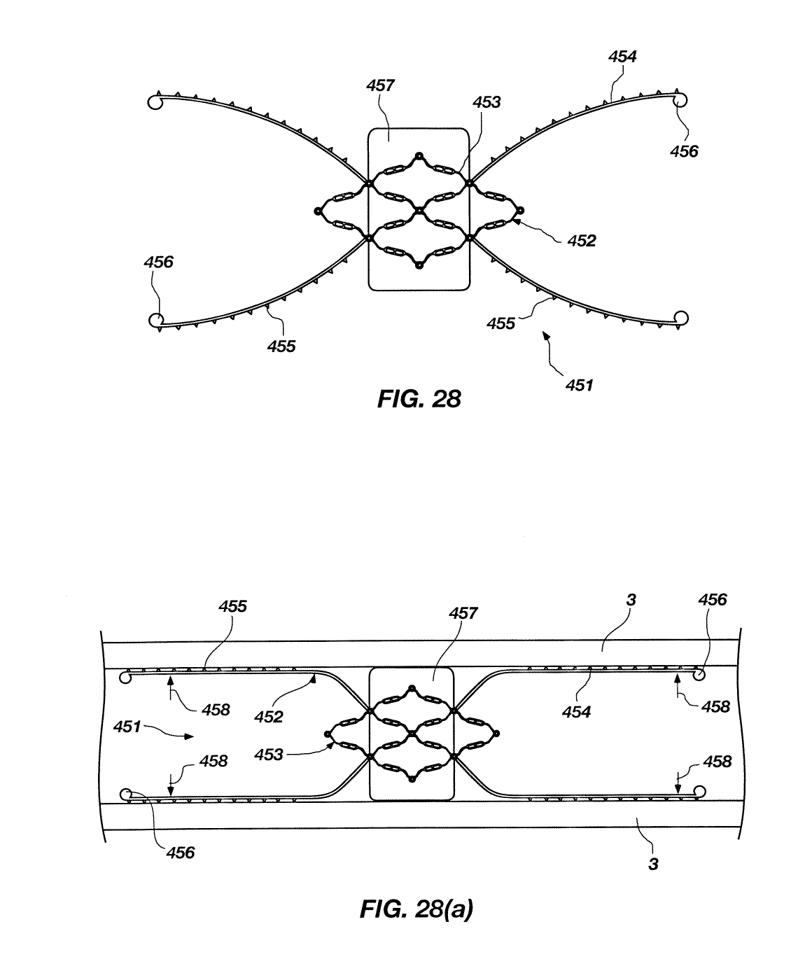

FIGS. 28 and 28(a) disclose another embodiment of a medical device 451, including similar features to the embodiments described with respect to FIGS. 20 and 21. The medical device 451 of this embodiment can include a frame member 452 with a central portion 453 having a multi-cellular structure and anchor members 454 with a tissue growth member 457 attached thereto. Further, the frame member can include a substantially flat or planar configuration configured to self expand from a narrow constrained position to a fully expanded position (as shown in FIG. 28). The anchors 454, however, in this embodiment, include anchors each having a single beam member. Such anchors 454 can extend distally and/or proximally. The anchors 454 comprising a single beam member can provide additional flexibility in the anchors while also conserving the constrained space within the tip of a catheter during delivery (not shown). The anchors 454 can include tines 455 extending laterally and outward sized and configured to provide resistance against tissue of the fallopian tube, thereby, preventing self migration of the medical device 451 and allowing the tissue growth member 457 to self-attach with tissue in-growth to the existing tissue of the fallopian tube. Further, the anchors 454 can include markers 456 at ends thereof. As illustrated in FIG. 28(a), there is disclosed the medical device 451 deployed in the fallopian tube 3 with the anchors 454 semi-constrained. As such, the anchors 454 can provide a biasing force 458 against the tissue of the fallopian tube 3 while the tissue growth member 457 occludes the fallopian tube.

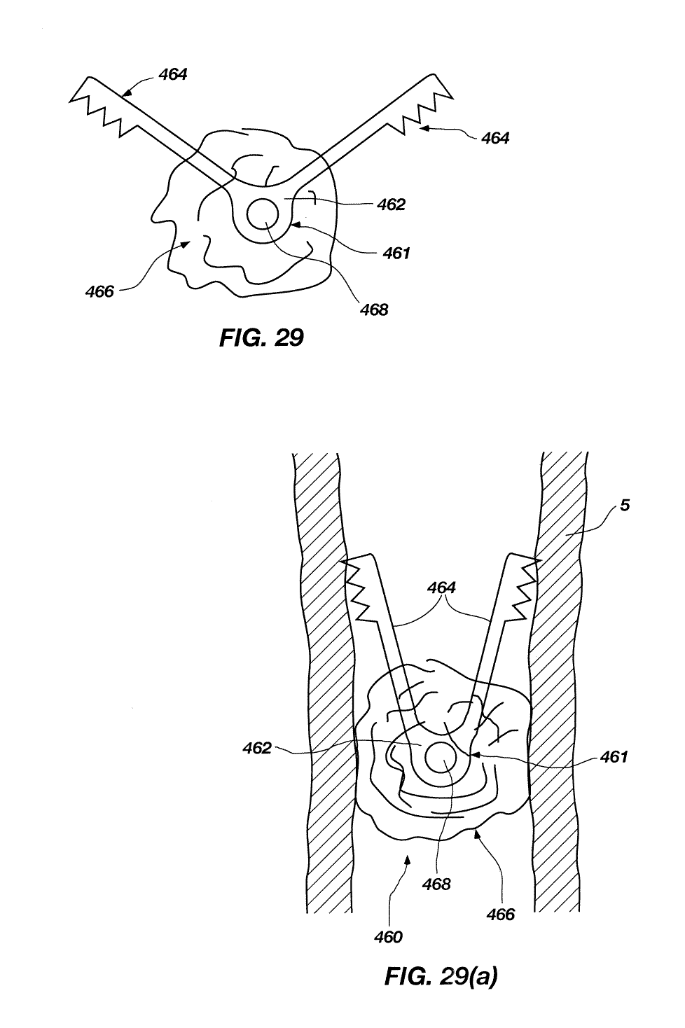

FIG. 29 discloses another embodiment of a medical device 460 configured to be positioned within a fallopian tube. In this embodiment, there is disclosed a frame 461 having central portion 462 with anchors 464 extending therefrom and a tissue growth member 466 disposed around the central portion 462 and a portion of the anchors 464. FIG. 29(a) discloses the medical device of FIG. 29 positioned within a fallopian tube 3. As illustrated, the anchor members 464 can be configured to engage the wall 5 of the fallopian tube 3 with the tissue growth member 466 stabilized in the tube. The tissue growth member 466 can be configured to expand and provide additional resistance to migration of the medical device within the fallopian tube. The central portion 462 can include a marker 468 to facilitate viewing and confirming position of the medical device 460 within the fallopian tube.

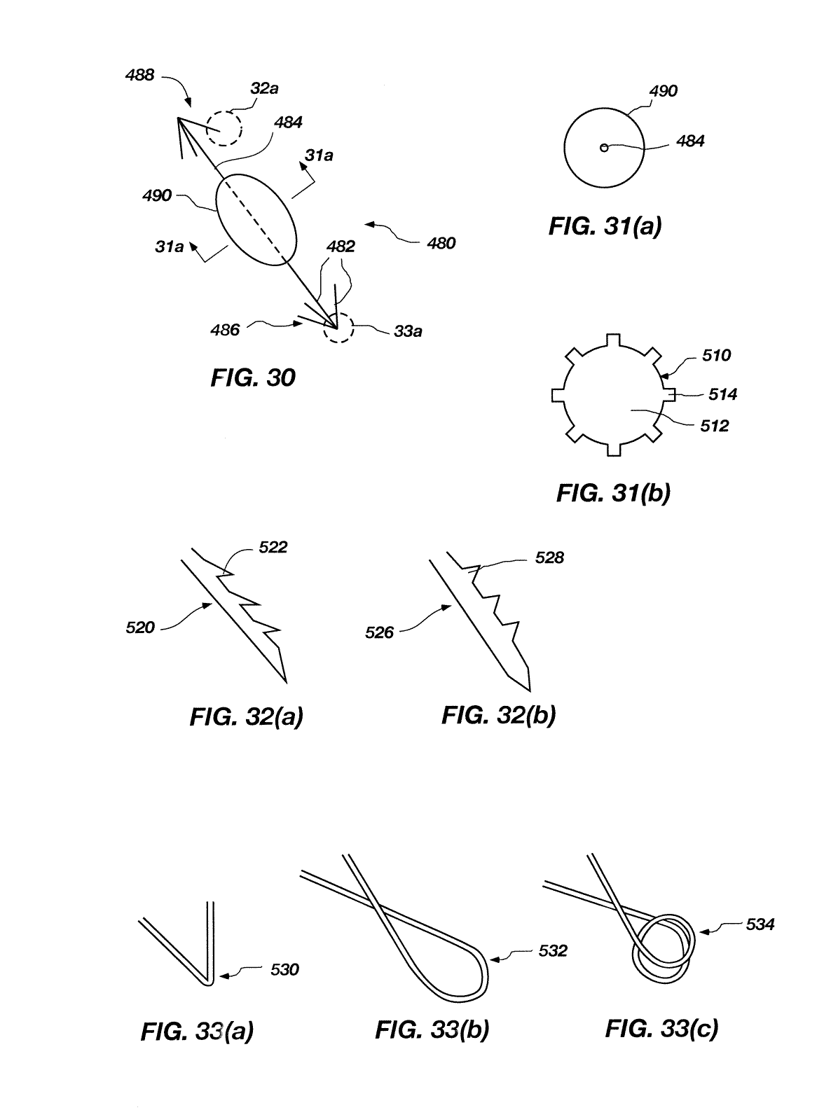

FIG. 30 discloses another embodiment of a medical device 480 for positioning and anchoring within a fallopian tube. In this embodiment, the medical device 480 can include a frame 482 having an elongated member 484 extending along an axis of the device and proximal anchors 486 and distal anchors 488 extending from ends of the elongated member 484. The medical device 480 can also include a tissue growth member 490 disposed around an intermediate portion of the elongated member 484. FIG. 31(a) discloses one embodiment of the tissue growth member 490, taken along line 31a. In this embodiment, the tissue growth member 490 can be circular in cross-section with the elongated member 484 extending through a central portion of the tissue growth member 490.

FIG. 31(b) discloses another embodiment of a tissue growth member 510 combining various features of previously described embodiments of tissue growth members. In particular, in this embodiment there is disclosed a tissue growth member 510 having a central portion 512 with a generally oval configuration and a circular cross-section with extension members 514 extending from the central portion 512. The extension members 514 can be configured to extend into the wall of the fallopian tube and grab or provide resistance to self-migration of the medical device within the fallopian tube.

FIGS. 32(a) and 32(b) disclose embodiments of anchor portions of the medical device, depicting an enlarged view taken from section 32a of FIG. 30. FIG. 32(a) discloses the anchor portions 520 having tines 522 extending therefrom and oriented distally of the anchor portion. The anchor portions 520 can also be oriented in a proximal direction of the anchor portions or both proximally and distally. FIG. 32(b) discloses the tines 528 oriented in a substantially outward or normal direction to the lengthwise direction of the anchor portion 526. It should also be noted that the anchor portions can be configured without tines, as depicted in FIG. 30.

FIGS. 33(a) through 33(c) disclose various embodiments of a junction between the anchor portions and the elongated member of the medical device, depicting an enlarged view taken from section 33a of FIG. 30. In one embodiment, as depicted in FIG. 33(a), the elongated member can include a folded portion 530 at an end of the elongated member to form the anchor portion. In another embodiment, the elongated member 484 can include a looped portion 532 to form the anchor portion, as depicted in FIG. 33(b). In still another embodiment, the elongated member can include a multi-looped configuration 534 to form the anchor portion, as depicted in FIG. 33(c). It can be determined which of such configurations to employ based on various considerations and factors, such as resistance, spring, manufacturing efficiency, cost, and sizing requirements. Further, as known to one of ordinary skill in the art, there are many other configurations and arrangements that can be employed for such anchor portions and the connection of such anchor portions to the elongated member 484.

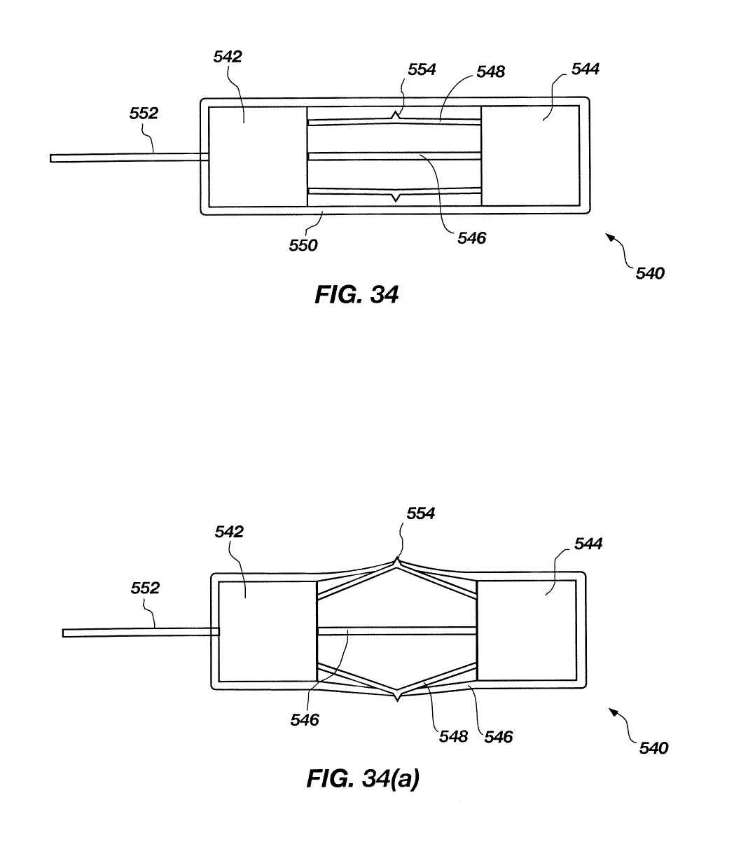

Referring now to FIGS. 34 and 34(a), there is disclosed another embodiment of a medical device 540 having a dual plug arrangement, according to the present invention. In particular, the medical device 540 of this embodiment can include a proximal plug 542 and a distal plug 544 with a guide member 546 and anchor members 548 therebetween. The distal plug 544 can be fixed to the guide member 546 and the proximal plug 542 can be configured to slide along the guide member 546 a predetermined distance via, for example, a stopper mechanism or ratchet type mechanism (not shown). Such proximal plug 542 can move along the guide member via a push member 552 being attached to the proximal plug 542. The anchor members 548 extend between the proximal and distal plugs 542 and 544 and are sized and configured to expand outward upon movement of the proximal plug 542 toward the distal plug 544. The anchor members 548 can extend within a substantially planar fashion (i.e., within a common plane) or can include additional anchor members extending in additional planes. Such anchor members 548 can include tines 554 that extend outward toward the tissue of the fallopian tube (not shown) and are configured to engage the fallopian tube. The medical device 540 can also include a tissue growth member 550. Such tissue growth member 550 can cover both the proximal and distal plugs 542 and 544 and extend over the anchor members 548 with the tines 554 extending through the tissue growth member 550. As such, the proximal plug 542 can be manually moved via the push member 552 a predetermined distance to expand the anchor members 548 into engagement with the tissue of the fallopian tube. With this engagement, the tissue growth member 550 also engages the tissue of the fallopian tube and, therefore, induces tissue growth thereto. In this manner, the proximal and distal plugs 542 and 544 and the tissue growth member 550 can provide initial occlusion of the fallopian tube as well as permanent sterilization.

As known by one of ordinary skill in the art, the various frame configurations that include self expanding configurations disclosed herein can be formed of Nitinol material, made from Nitinol sheets by laser cutting or any other suitable method, such as etching, or any other suitable manufacturing method. In addition, such frame configurations can be made to be manually self expanding, wherein such a frame can be formed of polymeric and/or metallic type material, such as polypropylene, polyester, PEEK, Teflon, titanium, stainless steel, copper or copper alloys, or any composite, combination or alloy thereof, or any other suitable material compatible with the human anatomy. Furthermore, such frame configurations and the tissue growth member can also be formed of a bio-resorbable material, such as polylactide, polyglycolide, poly-L-lactide, poly-DL-lactide, and various combinations thereof or any other suitable bio-resorbable material.

While the invention may be susceptible to various modifications and alternative forms, specific embodiments have been shown by way of example in the drawings and have been described in detail herein. However, it should be understood that the invention is not intended to be limited to the particular forms disclosed. Rather, the invention includes all modifications, equivalents, and alternatives falling within the spirit and scope of the invention as defined by the following appended claims. Furthermore, while the detailed description has disclosed systems, devices and methods for occluding a fallopian tube, the invention, as disclosed in the embodiments herein or any combinations/modifications thereof, can be employed to occlude other openings, ducts, tubes, or lumens within any suitable anatomy, such as enteric fistula, arterio-venous malformations, patent ductus arteriosis, patent foramen ovale, and left atrial appendage.

What is claimed is:

1. A medical device configured to occlude a fallopian tube, comprising:
an outer member having an outer surface and an inner surface, the inner surface defining a bore in the outer member;
an inner member configured to be positioned within the bore of the outer member; and
a tissue growth member attached to the outer surface of the outer member and configured to induce tissue growth within the fallopian tube to the outer member.

2. The medical device of claim 1, wherein the inner member is configured to provide a sealing fit with the outer member.

3. The medical device of claim 1, wherein the inner member is configured to be removable from the outer member to, thereby, expose the bore of the outer member.

4. The medical device of claim 1, further comprising at least one anchor extending from the outer member configured to anchor the medical device within the fallopian tube.

5. The medical device of claim 1, wherein the inner surface of the outer member comprises at least one protrusion configured to maintain the inner member from self migrating from the outer member.

6. The medical device of claim 1, wherein the inner member comprises an elastomeric material with at least one rigid rod at least partially disposed within the elastomeric material.

7. The medical device of claim 6, wherein the at least one rigid rod comprises a holding element configured to extend proximally from the elastomeric material and configured to assist removing the inner member from the outer member.

8. The medical device of claim 1, wherein the inner member comprises an outer surface defining ribs to provide a sealing fit within the bore of the outer member.

9. The medical device of claim 1, wherein the tissue growth member comprises a porous structure.

10. The medical device of claim 1, wherein the tissue growth member comprises foam.

11. The medical device of claim 1, wherein the tissue growth member comprises tines configured to anchor the device in the fallopian tube.

12. A method for reversing occlusion of a fallopian tube, the method comprising:
inserting a medical device having an outer member and an inner member into a fallopian tube;
anchoring the outer member of the medical device in the fallopian tube; and
partially removing the medical device from the fallopian tube by removing the inner member from the outer member to expose a bore in the outer member.

13. The method of claim 12, further comprising allowing tissue to grow to a tissue growth member attached to a periphery of the outer member of the medical device.

14. The method of claim 13, wherein the partially removing comprises:
extending a distal portion of a catheter in the fallopian tube against a proximal side of the outer member;
capturing a holding element extending proximally from the inner member with a capturing member disposed within the catheter; and
applying a biasing force against the proximal side of the outer member with the catheter and a pulling force on the holding element to remove the inner member from the outer member.

15. A medical device configured to be positioned within a fallopian tube of a human, the medical device comprising:
a frame member having a substantially flat configuration and configured to be moved between a narrow constrained position and an expanded position; and
a tissue growth member attached to the frame member and configured to induce tissue growth thereto.

16. The medical device of claim 15, wherein the tissue growth member is configured to expand out-of-plane from the substantially flat configuration of the frame member and configured to occlude the fallopian tube.

17. The medical device of claim 15, wherein the frame member comprises tines configured to assist preventing migration within the fallopian tube.

18. The medical device of claim 15, wherein the frame member comprises anchors configured to expand outward and bias against walls of the fallopian tube.

19. The medical device of claim 15, wherein the frame member is configured to self expand from the narrow constrained position to the expanded position.

20. The medical device of claim 15, wherein the frame member comprises a central portion having a multicellular structure configured to maintain the frame member in the substantially flat configuration.

21. A medical device configured to occlude a fallopian tube, comprising:
an expandable structure having a foam-like structure and configured to be movable between a contained position and an expanded position;
a marker coupled to the expandable structure; and
at least one anchor coupled to the expandable structure and configured to anchor the expandable structure within the fallopian tube.

22. The medical device of claim 21, wherein the expandable structure comprises a circular configuration.

23. The medical device of claim 21, wherein the expandable structure comprises an elongated expandable configuration.

24. The medical device of claim 21, wherein the at least one anchor comprises a plurality of micro tines unitarily formed with the expandable structure and extending from the expandable structure.

25. The medical device of claim 21, further comprising a frame including a central portion coupled to the expandable structure and including the at least one anchor, the at least one anchor extending from the central portion and the expandable structure and configured to anchor the expandable structure within the fallopian tube.
